(19) United States
(12) Patent Application Publication
(10) Pub. No.: US 2005/0273074 A1
(43) Pub. Date: Dec. 8, 2005

(54) Intravascular catheter delivery system

(76) Inventor: Joseph G. Lewis, Newtown Square, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/860,709

(22) Filed: Jun. 3, 2004

(51) Int. Cl. A61M 31/00

(52) U.S. Cl. 604/508; 604/264; 606/191

(57) ABSTRACT

Intravascular catheter assemblies are provided comprising multiple catheters for crossing areas of stenosis and providing conduits for intravascular delivery through and distal to the area of stenosis or otherwise of interest

### FIELD OF THE INVENTION

This invention is concerned with catheters for crossing areas of stenosis and providing conduits for intravascular delivery through and distal to the area of stenosis.

### BACKGROUND OF THE INVENTION

The delivery of vascular devices through areas of stenosis has been studied somewhat. Stenosis refers to an area of narrowing in a blood vessel. Such narrowing may range from the minimal to occlusion of the entire vessel lumen. Common sites of stenosis include the carotid and coronary arteries where such stenosis is usually caused by atherosclerosis.

In atherosclerosis, fat, cholesterol, and other substances build up in the inner lining of an artery. Such build-up is called an atheroma or plaque. Over time, the build-up results in stenosis of the vessel. Such stenosis results in decreased blood flow distal to the site of stenosis and can result in ischemia. In the heart, such ischemia may manifest itself in chest pain and myocardial infarction. In the brain, such ischemia may manifest itself as a stroke.

Most medical interventions in the treatment of these pathologies focus on the site of stenosis (e.g. surgically bypassing the stenosis through coronary bypass grafts; surgically removing the plaque causing the stenosis through carotid endarterectomy; percutaneously opening the stenosis through angioplasty and stent placement; percutaneously determining disease severity through catheterization and angiography). During these interventions, plaque and other debris may be released into the bloodstream and travel distal to the site of stenosis. Such release often causes ischemia and/or necrosis at another site and is referred to as an embolic event. The risk of such embolic events limits the usefulness of such procedures as this risk must be weighed against the risk of greater pathology without further treatment.

Recent advances and clinical trials focusing on carotid interventions such as stenting have created an even greater interest in limiting the risk of embolic complications which may cause a stroke and even death related to the procedure. Many researchers have attempted to limit the effects of such embolic events by designing protection devices such as nets, filters, and liners that “catch” or prevent the plaque or debris from traveling through the bloodstream and causing further ischemia. See, e.g., U.S. Pat. Nos. 6,582,448; 6,383,171. These efforts have had limited success. This limited success, in part, is because the protection device can itself cause an embolic event. Such an event may occur at the time of delivery or removal. Another significant limitation of such a protection device is operator difficulty advancing the device through the area of stenosis. In addition to limiting the success of the procedures, advancement difficulties may significantly increase patient exposure to anesthesia, radiation, and contrast agents and drain medical resources. This invention will improve the safety and efficacy of percutaneous interventions, particularly of the carotid and coronary arteries.

A safe, effective, and efficient system-for facilitating the delivery of such devices through and distal to an area of stenosis is needed. A system for delivering and removing other vascular devices including vascular instruments through the site of stenosis would also be useful. The present invention provides such a delivery system.

In accordance with one aspect of this invention, a catheter assembly comprising multiple catheters is used to cross the stenosis. This catheter assembly provides increased control and enables the user to use the traditional “push-pull” method of advancement. The push-pull technique involves retracting an inner catheter and wire while advancing an outer catheter(s) over the inner catheter and wire. The assembly of catheters of the invention facilitates its own delivery by its design. Such an embodiment enables controlled advancement of a catheter or device through and distal to an area of stenosis, thus decreasing the risk of an embolic event and the time necessary for the procedure. While other researchers have considered used telescoping catheters, these telescoping catheters were not designed to cross the stenosis, nor were they designed to actually facilitate their own delivery. See U.S. Pat. No. 5,120,323. In accordance with another aspect of the invention, the assembly of catheters may be used to deliver a device through and distal to an area of stenosis.

### SUMMARY OF THE INVENTION

In accordance with the objects of the invention, a catheter assembly is provided that is able to cross an area of stenosis and to provide a conduit for intravascular delivery. One of ordinary skill would understand that a delivered device may also be removed through the catheter assembly. One of skill in the art would also understand that there are multiple applications of such an invention. Furthermore, the invention may be used in many parts of the vasculature including the carotid and coronary arteries.

In one embodiment a catheter assembly of the present invention consists of three catheters—an inner, an intermediate, gradual, transition catheter, and a delivery catheter. In other embodiments a catheter assembly may consist of additional catheters. In some embodiments the leading aspects of these catheters are tapered at less than about a 45 degree angle from the centerline of the guide wire.

In addition to crossing an area of stenosis, in some embodiments a catheter assembly of the invention may be used as a conduit for intravascular delivery of devices through an area of stenosis. When a catheter assembly of the invention crosses an area of stenosis, a device may be inserted into the delivery catheter. The delivery catheter serves as a conduit for the device such as a stent, intravascular ultrasound catheter or distal protection device, enabling the device to pass through and distal to the stenosis. One of skill in the art will recognize that such a system limits the likelihood of device insertion related embolic events.

### BRIEF DESCRIPTION OF THE DRAWINGS

FIG. 1 is a sectional view of a catheter assembly of the present invention for crossing an area of stenosis and providing a conduit for intravascular delivery or removal of devices through an area of stenosis.

FIG. 2 is a sectional view of a catheter according to one embodiment of the invention.

FIG. 3 is a sectional view of a catheter according to another embodiment of the invention.

FIG. 4 is a sectional view of a catheter according to yet another embodiment of the invention.

This invention relates to a catheter assembly for crossing areas of stenosis and for intravascular delivery or removal of devices through areas of stenosis. The present invention may be used in the coronary arteries, carotid arteries, renal arteries, saphenous veins, bypass grafts, and other natural and artificial vessels. The present invention may be used alone or in conjunction with another procedure (e.g. catheterization, angioplasty, stenting). While directed to crossing an area of stenosis, an assembly of the invention may also be used in vessel areas conventional catheters have difficulty crossing e.g. areas of tortuosity.

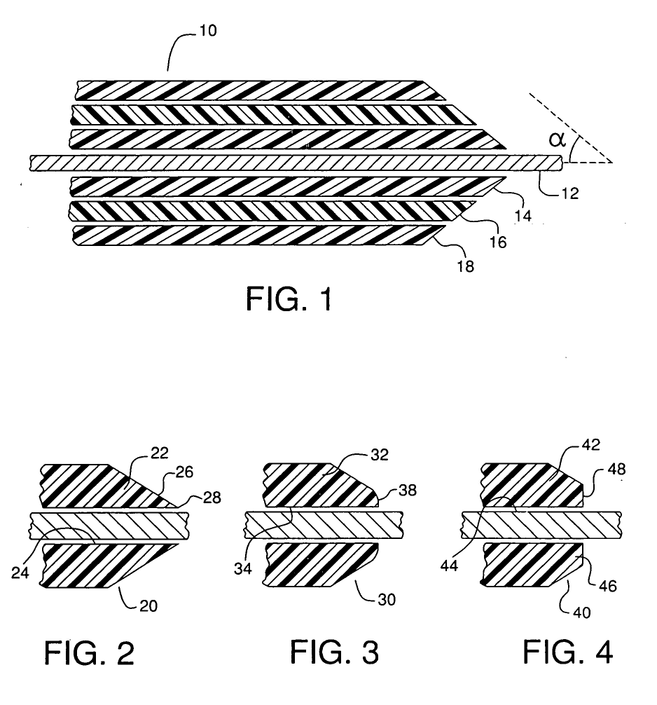

As shown in FIG. 1 a catheter assembly is indicated by numeral 10 and comprises an inner catheter 14 slidable on a guide wire 12, at least one intermediate gradual transition catheter 16 slidable on an inner catheter, and a delivery catheter 18 slidable on the outermost intermediate gradual transition catheter. As will be described more fully hereinafter, the distal portions of the catheters may form an angle with the guide wire. This angle, conveniently denominated, α, is measured between the center line of the guide wire when a catheter is in place thereupon and the leading edge of the catheter.

A catheter assembly of the invention may be inserted over a guide wire. A guide wire may be constructed by methods known in the art. The diameter of said guide wire may range from about 0.010-0.038 inches, preferably, 0.014 inches in coronary artery applications, and from about 0.014-0.035 inches in carotid applications. A person of ordinary skill will recognize that guide wire and catheter selection is dependent on multiple factors including vessel size; operator preference and so forth.

The catheters may be constructed by methods known in the art. In some embodiments, the catheters are comprised of polymer and are elongated tubular structures. In some preferred embodiments at least one catheter of the assembly may be comprised of a polyurethane and have a hydrophilic outer coating. In some preferred embodiments at least one catheter of the assembly is comprised of polytetrafluoroethylene. The precise manufacture and coating of a guide wire or individual catheter is not part of the invention. It is within skill of persons in the art.

Selection of polymer makeup and coating composition is dependent to a degree upon whether a catheter will function as an inner, intermediate gradual transition or delivery catheter. For example, in some embodiments an inner catheter with greater rigidity to enable the operator to use the push-pull method may be preferred. Additionally, a delivery catheter needs to be flexible enough to track through the vessel of interest but rigid enough to avoid kinking and allow delivery of the device. The degree of flexibility or rigidity for any catheter may be altered by varying the materials constructing the catheter by methods known in the art.

Catheter diameter, either inner or outer, will be influenced by multiple factors including the vessel involved; the device to be delivered; tortuouosity of the introduction path; vessel calcifications; difficulty delivering the device and so forth. Inner diameter refers to the diameter of the catheter lumen. Outer diameter refers to the full thickness of the catheter (includes the inner diameter). In some embodiments it will be preferred to minimize each catheter diameter, either inner or outer or both, while retaining the functional use of the invention. For example, in some embodiments the inner diameter of the delivery catheter would be just large enough to allow an intravascular device to be advanced through it. Either inner or outer diameter size of any catheter may be selected to allow passage of the assembly through and distal to the stenosis or passage of an intravascular device through and distal to the stenosis.

In some embodiments the inner catheter inner diameter will be in the range from about 0.010-0.021 inches and outer diameter will be in the range from about 0.03-0.04 inches. In some preferred embodiments, the inner catheter inner diameter will be about 0.014 inches and the outer diameter about 0.033 inches. The intermediate, gradual transition catheter is designed to provide a relatively smooth and effectively seamless transition between inner catheter and delivery catheter. The intermediate gradual transition catheter inner diameter is dependent on the inner catheter outer diameter and the intermediate gradual transition catheter outer diameter is dependent on the delivery catheter inner diameter. The delivery catheter inner diameter is designed to allow delivery of a device. Therefore, the delivery catheter inner diameter is dependent on the device to be delivered, and in-some embodiments will be in the range from about 0.030-0.078 inches, preferably 0.039-0.052 inches.

Referring to FIG. 2, a catheter of the present invention is generally given the reference number 20. It is understood that the catheter 20 may be an inner catheter, such as described in FIG. 1 with reference to inner catheter 14, an intermediate gradual transition catheter, such as described in FIG. 1 with reference to intermediate gradual transition catheter 16, or a delivery catheter, such as described in FIG. 1 with reference to delivery catheter 18.

The catheter 20 comprises a distal tip portion 22. An interior wall 24 is defined by the catheter 20. A tapering leading aspect 26 is disposed externally to the interior wall 24, terminating in a tip edge 28. In this embodiment, the tip edge 28 comes to a sharp point.

Angle α between the interior wall 24 or centerline of the guidewire and the tapering leading aspect 26 of the catheter 20, in one embodiment, is in a range from about 10 degrees to about 45 degrees. In another embodiment, α is in a range from about 20 degrees to about 35 degrees. Preferably, α is less than about 35 degrees.

Referring to FIG. 3, in another embodiment, a catheter of the present invention is generally given the reference number 30. It is understood that the catheter 30 may be an inner catheter, such as described in FIG. 1 with reference to inner catheter 14, an intermediate gradual transition catheter, such as described in FIG. 1 with reference to intermediate gradual transition catheter 16, or a delivery catheter, such as described in FIG. 1 with reference to delivery catheter 18.

The catheter 30 comprises a distal tip portion 32. An interior wall 34 is defined by the catheter 30. A tapering leading aspect 36 is disposed externally to the interior wall 34, terminating in a tip edge 38. In this embodiment, the tip edge 38 comes to a rounded point.

Angle α defined between the interior wall 34 or centerline of the guidewire and the tapering leading aspect 36 of the catheter 30, in one embodiment, is in a range from about 10 degrees to about 45 degrees. In another embodiment, α is in a range from about 20 degree to about 35 degrees. Preferably, α is less than about 35 degrees.

Referring to FIG. 4, in another embodiment, a catheter of the present invention is generally given the reference number 40. It is understood that the catheter 40 may be an inner catheter, such as described in FIG. 1 with reference to inner catheter 14, an intermediate gradual transition catheter, such as described in FIG. 1 with reference to intermediate gradual transition catheter 16, or a delivery catheter, such as described in FIG. 1 with reference to delivery catheter 18.

The catheter 40 comprises a distal tip portion 42. An interior wall 44 is defined by the catheter 40. A tapering leading aspect 46 is disposed externally to the interior wall 44, terminating in a tip edge 48. In this embodiment, the tip edge 48 comes to a blunt point.

Angle α, defined between the interior wall 44 or guidewire centerline and the tapering leading aspect 46 of the catheter 40 is in a range from about 10 degrees to about 45 degrees. In another embodiment, α is in a range from about 20 degree to about 35 degrees. Preferably, α is less than about 35 degrees.

The tip of each catheter may be constructed by methods known in the art. In some embodiments the leading tip may be constructed from a polymer blend, preferably comprising a polyurethane. The tip may also comprise a hydrophilic coating. One of skill in the art will recognize that the catheter tip may be manufactured at the time of catheter manufacture or separately.

A catheter tip is designed to enable the catheter to ease across the stenosis with a minimum of trauma to the vessel or the area of stenosis. The tip is angled to further this crossing. In some embodiments, each catheter tip angle α is less than about 45 degrees, preferably less than about 35 degrees, and preferably between about 20-35 degrees. One of skill in the art will recognize that in some embodiments it is preferable that the catheter tip angles of at least two catheters be within about 5 degrees of each other. In some preferred embodiments the catheter tip angle of all catheters may be the same.

Catheter assembly may be modified by other methods known in the art, e.g. the tip or part of at least one catheter may be radioopaque or a catheter may have side holes to allow for distal perfusion.

Catheter length may also be varied. In some embodiments, the catheters may be designed so that a catheter leads another catheter. For example, in some embodiments it is preferred for the inner catheter to lead the next intermediate gradual transition catheter by about 5-20 inches, preferably about 10-15 inches. In some embodiments it may be preferred for the intermediate gradual transition catheter to lead a delivery catheter by about 1 inch. In embodiments wherein the inner catheter leads an intermediate gradual transition catheter the inner catheter will cross the stenosis before the intermediate gradual transition catheter. In embodiments wherein an intermediate gradual transition catheter leads a delivery catheter the intermediate gradual transition will cross the stenosis before the delivery catheter.

Each catheter for a catheter assembly may be constructed separately and then combined together to form a catheter assembly. The catheters may be assembled by sliding an intermediate gradual transition catheter over the inner catheter and a delivery catheter over an intermediate gradual transition catheter. There may be more than one intermediate gradual transition or delivery catheter. Proximally, these catheters are connected via a hub which allows them to be advanced together as a unit but will allow them be separately advanced one over the other when necessary. Additionally, in some embodiments at least one intermediate gradual transition catheter or at least one delivery catheter may be removed and an alternative catheter substituted. For example, this substitution may be performed by the operator at the time of the intervention. In some embodiments such substitution may be performed to enable the delivery of an additional device or removal of a device without recrossing the stenosis with an additional catheter assembly of the invention.

To use the invention, an operator may procede according to methods known in the art to access the vessel containing the stenosis. For example, in a coronary intervention the operator may advance the guiding catheter of choice to the ostium of the left main coronary artery or right coronary artery. In a carotid application, an operator may advance a sheath into the common carotid artery. The operator could then pass a guide wire across the area of stenosis. After passing the guide wire, the operator could place the catheter assembly on the wire. The catheter assembly is loaded on the proximal end of the guidewire and advanced as a unit. The innermost catheter may be advanced over the guide wire and through the stenosis reaching a point distal to the stenosis. In some embodiments it is preferred that the innermost catheter reach a point just distal to the stenosis. In other embodiments it is preferred that the innermost catheter reach a point at least about 10-20 mm distal to the stenosis. An intermediate gradual transition catheter and delivery catheter may then be advanced over the inner catheter and through the stenosis so that each also reaches a point distal to the stenosis, preferably at least about 5-10 mm distal to the stenosis. One of ordinary skill will recognize that catheters of the catheter assembly may reach different points distal to the stenosis.

More than one intermediate gradual transition catheter may be used although one of ordinary skill would understand that successive intermediate gradual transition catheters would advance over an intermediate gradual transition catheter rather than an inner catheter. While the guide wire may be exchanged for an alternative wire at any point during the procedure, it may be preferred in some embodiments of the invention or by some operators to exchange the first guide wire for an alternative, more rigid, guide wire after an inner catheter had crossed the area of stenosis. One of ordinary skill will recognize that certain techniques and catheter skills known in the art may be used at any stage of the procedure such as easing advancement by holding the wire and inner catheter stationary or using the push-pull technique.

After the delivery catheter has been advanced to a point distal to the stenosis, any intermediate gradual transition and inner catheters may be removed. The guide wire may or may not remain depending on multiple-factors e.g. vessel, intervention, or device to be delivered. For example, for a delivery of a carotid artery filter device, one would remove the guide wire before inserting the filter wire. In many coronary artery applications, the guide wire does not need to be removed.

After removal of inner and intermediate gradual transition catheters an intravascular device may be inserted into and through the delivery catheter until it exits the delivery catheter distal to the stenosis. Intravascular devices that may be used include, but are not limited to, the following: a stent, a distal protection device or filter, an intravascular ultrasound, and so forth.

One of skill in the art will recognize that an inserted intravascular device may also be removed through the delivery catheter. Any remaining catheters and the guide wire may be removed in accordance with methods known in the art.

1. An intravascular catheter assembly comprising:
an inner catheter slidable on a guide wire;
an intermediate gradual transition catheter slidable on the inner catheter; and
a delivery catheter slidable on the intermediate gradual transition catheter; each catheter having a distal end for insertion into the vasculature of a patient and through an area of stenosis, the leading aspect of each catheter having a taper at an angle less than about 35 degrees with respect to the horizontal axis.

2. The assembly of claim 1 wherein at least one catheter is comprised of a polyurethane.

3. The assembly of claim 1 wherein at least one catheter is comprised of a polytetrafluoroethylene and a hydrophilic coating.

4. The assembly of claim 1 wherein at least two catheter angle tips are tapered within 5 degrees of one another.

5. The assembly of claim 1 wherein at least two catheter angle tips are tapered within 1 degree of one another.

6. The assembly of claim 1 wherein at least a portion of each catheter has a hydrophilic outer coating.

7. The assembly of claim 1 wherein at least two catheter angle tip edges are the same.

8. The assembly of claim 1 wherein at least one catheter diameter of the catheter assembly is selected to allow the assembly to pass through and distal to an area of stenosis.

9. The assembly of claim 1 wherein at least one catheter diameter of the catheter assembly is selected to allow an intravascular device to pass through and distal to an area of stenosis.

10. The assembly of claim 1 wherein catheter assembly is used to deliver an intravascular device across an area of vessel tortuosity.

11. A method of delivering an intravascular device through an area of stenosis comprising:
passing a guide wire across stenosis;
passing an inner catheter on guide wire across stenosis;
passing an intermediate gradual transition catheter across stenosis;
passing a delivery catheter across stenosis; and
passing an intravascular device through the delivery catheter and across the stenosis.

12. The method of claim 11 wherein at least one catheter leads another catheter.

13. The method of claim 11 wherein an inner catheter leads an intermediate gradual transition catheter that leads a delivery catheter.

14. The method of claim 11 wherein an intermediate gradual transition catheter and delivery catheter are passed across the stenosis concurrently.

15. The method of claim 11 wherein an intermediate gradual transition catheter and delivery catheter are passed across the stenosis as a unit.

16. The method of claim 11 wherein the inner catheter is fixed distal to the stenosis.

17. The method of claim 11 wherein at least one catheter is advanced using the push-pull technique.

18. The method of claim 11 wherein at least one catheter is removed before said intravascular device is advanced.

19. The method of claim 11 wherein at least one catheter diameter of the catheter assembly is selected to allow the assembly to pass through and distal to an area of stenosis.

20. The method of claim 11 wherein at least one catheter diameter of the catheter assembly is selected to allow a device to pass through and distal to an area of stenosis.

21. A method of delivering an intravascular device across an area of vessel tortuosity comprising:
passing a guide wire across tortuosity;
passing an inner catheter on guide wire across tortuosity;
passing an intermediate gradual transition catheter across tortuosity;
passing a delivery catheter across tortuosity; and
passing an intravascular device through the delivery catheter and across the tortuosity.
